SEQUENCE LISTING (12 sequences)

SEQ ID NO: 1 - protein, 170 aa, Homo sapiens
MDTRNKAQLLVLLTLLSVLFSQTSAWPLYRAPSALRLGDRIPFEGANEPDQVSLKEDIDMLQNALAENDTPYYDVSRNARHADGVFTSDFSKLLGQLSAKKYLESLMGKRVSSNISEDPVPVKRHSDAVFTDNYTRLRKQMAVKKYLNSILNGKRSSEGESPDFPEELEK

SEQ ID NO: 2 - protein, 28 aa, Homo sapiens
HSDAVFTDNYTRLRKQMAVKKYLNSILN

SEQ ID NO: 3 - DNA, 1601 bp, Homo sapiens
agggtagagtgagaagcaccagcaggcagtaacagccaacccttagccattgctaagggcagagaactggtggagcctttctcttactcccaggacttcagcacctaagacagctccaaaacaaaccagaacagtcagctccgggggagcacgactgggcgagaggcacagaaatggacaccagaaataaggcccagctccttgtgctcctgactcttctcagtgtgctcttctcacagacttcggcatggcctctttacagggcaccttctgctctcaggttgggtgacagaataccctttgagggagcaaatgaacctgatcaagtttcattaaaagaagacattgacatgttgcaaaatgcattagctgaaaatgacacaccctattatgatgtatccagaaatgccaggcatgctgatggagttttcaccagtgacttcagtaaactcttgggtcaactttctgccaaaaagtaccttgagtctcttatgggaaaacgtgttagcagtaacatctcagaagaccctgtaccagtcaaacgtcactcagatgcagtcttcactgacaactatacccgccttagaaaacaaatggctgtaaagaaatatttgaactcaattctgaatggaaagaggagcagtgagggagaatctcccgactttccagaagagttagaaaaatgatgaaaaagacctttggagcaaagctgatgacaacttcccagtgaattcttgaaggaaaatgatacgcaacataattaaattttgagttctacataagtaattcaagaaaacaacttcaatatccaaaccaaataaaaatattgtgttgtgaatgttgtgatgtattctagctaatgtaataactgtgaagtttacattgtaaatagtatttgagagttctaaattttgtctttaactcataaaaagcctgcaatttcatatgctgtatatcctttctaacaaaaaaatatatttaatgataagtaaatgctaggttaattccaattatatgagacgtttttggaagagtagtaatagagcaaaattgatgtgtttatttatagagtgtacttaactattcaggagagtagaacagataatcagtgtgtctaaatttgaatgttaagcagatggaatgctgtgttaaataaacctcaaaatgtctaagatagtaacaatgaagataaaaagacattcttccaaaaagattttcagaaaatattatgtgtttccatattttataggcaacctttatttttaatggtgttttaaaaaatctcaaatttggattgctaatcaccaaaggctctctcctgatagtctttcagttaaggagaacgacccctgcttctgacactgaaacttccctttctgcttgtgttaagtatgtgtaaaatgtgaagtgaatgaaacactcagttgttcaataataaatatttttgccataatgactcagaatattgctttggtcatatgagcttccttctgtgaaagtacatttggagacacaactatttttccaaaataattttaagaaatcaaagagagaaaataaagaccttgcttatgattgcagataaaaaaaaaaaaaaaaaa

SEQ ID NO: 4 - protein, 169 aa, Homo sapiens
MDTRNKAQLLVLLTLLSVLFSQTSAWPLYRAPSALRLGDRIPFEGANEPDQVSLKEDIDMLQNALAENDTPYYDVSRNARHADGVFTSDFSKLLGQLSAKKYLESLMGKRVSNISEDPVPVKRHSDAVFTDNYTRLRKQMAVKKYLNSILNGKRSSEGESPDFPEELEK

SEQ ID NO: 5 - protein, 28 aa, Homo sapiens
HSDAVFTDNYTRLRKQMAVKKYLNSILN

SEQ ID NO: 6 - DNA, 1598 bp, Homo sapiens
agggtagagtgagaagcaccagcaggcagtaacagccaacccttagccattgctaagggcagagaactggtggagcctttctcttactcccaggacttcagcacctaagacagctccaaaacaaaccagaacagtcagctccgggggagcacgactgggcgagaggcacagaaatggacaccagaaataaggcccagctccttgtgctcctgactcttctcagtgtgctcttctcacagacttcggcatggcctctttacagggcaccttctgctctcaggttgggtgacagaataccctttgagggagcaaatgaacctgatcaagtttcattaaaagaagacattgacatgttgcaaaatgcattagctgaaaatgacacaccctattatgatgtatccagaaatgccaggcatgctgatggagttttcaccagtgacttcagtaaactcttgggtcaactttctgccaaaaagtaccttgagtctcttatgggaaaacgtgttagtaacatctcagaagaccctgtaccagtcaaacgtcactcagatgcagtcttcactgacaactatacccgccttagaaaacaaatggctgtaaagaaatatttgaactcaattctgaatggaaagaggagcagtgagggagaatctcccgactttccagaagagttagaaaaatgatgaaaaagacctttggagcaaagctgatgacaacttcccagtgaattcttgaaggaaaatgatacgcaacataattaaattttgagttctacataagtaattcaagaaaacaacttcaatatccaaaccaaataaaaatattgtgttgtgaatgttgtgatgtattctagctaatgtaataactgtgaagtttacattgtaaatagtatttgagagttctaaattttgtctttaactcataaaaagcctgcaatttcatatgctgtatatcctttctaacaaaaaaatatatttaatgataagtaaatgctaggttaattccaattatatgagacgtttttggaagagtagtaatagagcaaaattgatgtgtttatttatagagtgtacttaactattcaggagagtagaacagataatcagtgtgtctaaatttgaatgttaagcagatggaatgctgtgttaaataaacctcaaaatgtctaagatagtaacaatgaagataaaaagacattcttccaaaaagattttcagaaaatattatgtgtttccatattttataggcaacctttatttttaatggtgttttaaaaaatctcaaatttggattgctaatcaccaaaggctctctcctgatagtctttcagttaaggagaacgacccctgcttctgacactgaaacttccctttctgcttgtgttaagtatgtgtaaaatgtgaagtgaatgaaacactcagttgttcaataataaatatttttgccataatgactcagaatattgctttggtcatatgagcttccttctgtgaaagtacatttggagacacaactatttttccaaaataattttaagaaatcaaagagagaaaataaagaccttgcttatgattgcagataaaaaaaaaaaaaaaaaa

SEQ ID NO: 7 - protein, 171 aa, Mus musculus
MEARSKPQFLAFLILFSVLFSQSLAWPLFGPPSVVSRLDDRMPFEGAGDPDQVSLKADSDILQNPLAENGTPYYDVSRNARHADGVFTSDYSRLLGQISAKKYLESLIGKRISSSISEDPVPIKRHSDAVFTDNYTRLRKQMAVKKYLNSILNGKRSSEGDSADFLEELEK

SEQ ID NO: 8 - protein, 28 aa, Mus musculus
RHSDAVFTDNYTRLRKQMAVKKYLNSIL

SEQ ID NO: 9 - DNA, 1527 bp, Mus musculus
agcttggacagcagagcactagccagctacagccaaccgttccccaggaaccgggaacagactggtggagccttccctagagcagaacttcagcaccctagacagctgccacgaagccggaaaggcagccctgcctgaaggaaacagccaaggaggcaccgagatggaagccagaagcaagcctcagttcctggcattcctgatactcttcagtgtgctgttctctcagtcgctggcctggcctctctttggaccaccttctgtagtgagtaggctggatgacaggatgccgtttgaaggagcaggtgaccctgaccaagtctctttaaaagcagactctgacatcttgcagaatcccttagcagaaaatggcacaccctattatgatgtgtcaagaaatgccaggcatgctgatggagttttcaccagcgattacagcagacttctgggtcagatttctgccaaaaaataccttgagtcactcattggcaaacgaatcagcagcagcatctcggaagatcctgtgccaatcaaacgacactctgatgccgtcttcacagataactacacccgcctcagaaagcaaatggctgtgaagaaatacctgaactccatcctgaatggaaagaggagcagtgagggagattctgcagactttcttgaagagctggagaaatgatgggaagaggcctctgggcagagctgaaatcagagaattctcgaaggaaaacaaccacgtgattacattatgagttctacatgtctaattcaagaaaaaaacttccatagcaaaaccaaataaaatgtgttgtgaatattgtggtttcctttatgtaataactgtgatgtttacattgtaaatattatttgagcattctaacattcatctgtagctcatgaaatgcttataatttcatatgctatatattctttcaaagaaaagtatatttaatgataggtagatactagattaattgcaattatctgaagctttctgcaagggtagcaatcgaggaaaattgatgtgtttatttatagcatgtagttaactattcaacagagcagaacagataatcagtgtgaacaagtctaaatgctaagcagataggctgctgtgttacataaggcaaaatatctaaggggaataacaaattatggataaaagagatatgtggcaaaaggattttcagaattgtatttctccagtgataggtactccatctctcacggattcatctctcccattaggctttgcaatccccaaaggctacttcagagatgcttcagctaggaaaagcccatcgtccaatctggggcttcccctttctgcgtgtgctatggatgtgtaaactagaagctaaatggagtgcttgatttccagtagtaaatacttctcccatagtcactcacaatgatattttgtcttattggcttcctttgctgaaagtacatttgtagacacaactatttttccaatgtgattgtatgaaattaaagacaggaataaagatctttggttatcattgc

SEQ ID NO: 10 - protein, 170 aa, Rattus norvegicus
MESRSKPQFLAILTLFSVLFSQSLAWPLYGPPSSVRLDDRLQFEGAGDPDQVSLKADSDILQNALAENDTPYYDVSRNARHADGVFTSDYSRLLGQISAKKYLESLIGKRISSSISEDPVPVKRHSDAVFTDNYTRLRKQMAVKKYLNSILNGKRSSEGDSPDFLEELEK

SEQ ID NO: 11 - protein, 28 aa, Rattus norvegicus
HSDAVFTDNYTRLRKQMAVKKYLNSILN

SEQ ID NO: 12 - DNA, 1532 bp, Rattus norvegicus
ctagcggctactgccaacctttccccaggaccaggggcagactccgtggagccttctcccaagcagaacttcagcaccccagacagctcccccgcgccggagagacggtcctgccagaaggaaagacccaaggaggcaccgagatggaatccagaagcaagcctcagttcctggcgatcctgacactcttcagtgtgctgttctcacagtcgctggcctggcctctctatgggccaccttcttcagtgaggttggatgacaggctgcagttcgaaggagcaggtgaccctgatcaagtctctttaaaagcagactctgacatcttgcagaatgccttagcggagaatgacacgccctattatgatgtgtccagaaatgccaggcatgctgatggagttttcaccagcgactacagtagacttctgggtcagatttctgccaaaaaataccttgagtcactcattggcaaacgaatcagcagtagcatctcggaagaccccgtgccggtcaaacgacactctgatgcagtcttcacagataactacacccgccttagaaagcaaatggctgtgaagaaatacttgaactccattctaaatgggaagaggagcagtgagggagattctccagacttccttgaagagctagagaaatgatgagaagggtcctctgggcagagctgaagatcagagaattcttgaaggaaaacaaccaagtgattacattatgagttctacatatctaattcaagaaaacaacttccatagcaaaaccaaataaaatgtgttgtgaatattgtggtttcctttatgtaataactgtgatgtttacattgtaaatatatttagcactctaaaattcatctttagctcgtgaaaggcttataatttcatatgctatatattctttaaaaaatatatttaatgataggtagatactagattaattgcaattatctgaagctttctgcaagggtagcaatcgaggaaaattgatgggcttatttatagcatgcagttaactattcaacagagcagaacagataatcagtgtgaccaagtctgaatgctaagcagataggctgccgtgttacataaagcaaaatatctaagggaaaaccaaacatatggaaaatggagatacttgacaaaaggattttcaaaattgtattcctccagtgatagggactccacctctcatggattcatctctccgactaggatttgcaatccccaaaagcttcttcgagttgcttcagctaggaaaagctcaacttccaacctggagcttccccttcctgcttgtgctgtggatgtgtaagctagaagcctaacggagtgcttgatttccagtagtaaatactctttccgtaatcactcacaacagtattttgtcttattggcttcctttgctgaaagtacatttgtagacacaactatttttccaatgtgattgtatgaaattaaagacaggaataaagatctttggttatcattgcaaaaaaaaaaaaaaaaaaaaaaaaaaaaaaaaaa

(19) United States
(12) Patent Application Publication
(10) Pub. No.: US 2016/0271224 A1
(43) Pub. Date: Sep. 22, 2016

(54) METHODS OF REDUCING CORNEAL ENDOTHELIAL CELL LOSS

(71) Applicants: MASSACHUSETTS EYE AND EAR INFIRMARY, Boston, MA (US); SCHEPENS EYE RESEARCH INSTITUTE, Boston, MA (US)

(72) Inventors: Takefumi Yamaguchi, Tokyo (JP); Ula Jurkunas, Winchester, MA (US); Pedram Hamrah, Wellesley, MA (US)

(21) Appl. No.: 15/037,292

(22) PCT Filed: Nov. 20, 2014
(86) PCT No.: PCT/US2014/066551

§ 371 Date: May 17, 2016

Related U.S. Application Data
(60) Provisional application No. 61/906,723, filed on Nov. 20, 2013.

(51) Int. Cl. A61K 38/22; A61B 3/10; A61K 9/00

(52) U.S. Cl. CPC A61K 38/2278; A61K 9/0048; A61B 3/1025

(57) ABSTRACT

Provided herein are methods of reducing corneal endothelial cell loss (e.g., nerve loss-related corneal endothelial cell loss) that include selecting a subject identified as having an eye with reduced numbers of corneal nerves as compared to a reference eye, e.g., an eye of a healthy control, and administering vasoactive intestinal peptide (VIP) or a nucleic acid encoding VIP to the selected subject.

### CROSS-REFERENCE TO RELATED APPLICATIONS

This application claims priority to U.S. Provisional Patent Application No. 61/906,723, filed on Nov. 20, 2013, the contents of which are incorporated herein by reference in their entirety.

### BACKGROUND OF THE INVENTION

The cornea is the most densely innervated tissue in the mammalian body. Intact innervation of the cornea is necessary for the maintenance of corneal structure and function (Araki et al., Curr. Eye. Res. 13:203-211, 1994; Nishida et al., Curr. Opin. Ophthalmol. 20:276-281, 2009). Corneal nerves can be damaged due to many pathological conditions, such as, e.g., ocular infection, surgery, diabetes, stroke, dry eye syndrome, and intracranial lesion involving the trigeminal nerve, which all result in complete or partial neurotrophic keratopathy. A latent nerve density decrease and concomitant endothelial cell loss is observed in various kinds of ocular pathology, including non-inflammatory chronic diseases, such as Fuchs' endothelial corneal dystrophy (Hoesl et al., Eye 27:42-49, 2013). Corneal nerve loss or damage also plays or is thought to play a role in pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, and graft rejection

### SUMMARY OF THE INVENTION

The invention is based, in part, on the discovery that administration of vasoactive intestinal peptide (VIP) decreases corneal endothelial cell loss (e.g., nerve loss-related corneal endothelial cell loss) in a mammal. In view of this discovery, provided herein are methods of reducing corneal endothelial cell oss (e.g., nerve-loss related corneal endothelial cell loss) in a subject that include selecting a subject identified as having an eye with reduced numbers of corneal nerves as compared to a reference eye, e.g., an eye of a healthy control, and administering VIP or a nucleic acid encoding a VIP to the selected subject. Also provided are pharmaceutical compositions that include VIP and one or more additional agents for treating Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection.

Provided herein are methods of reducing nerve loss-related corneal endothelial cell loss in a subject that include selecting a subject identified as having an eye with reduced numbers of corneal nerves as compared to a reference eye, e.g., an eye of a healthy control, and administering vasoactive intestinal peptide (VIP) to the selected subject. In some embodiments of any of the methods described herein, the VIP is topically administered to the eye of the subject. In some embodiments of any of the methods described herein, the VIP is administered to the eye of the subject by systemic administration, subconjunctival injection, or intraperitoneal injection.

In some embodiments of any of the methods described herein, the subject has Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection. In some embodiments of any of the methods described herein, the subject has been diagnosed as having Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection. In some embodiments of any of the methods described herein the subject has and/or has been diagnosed as having Fuchs' endothelial corneal dystrophy.

Some embodiments of any of the methods described herein further include identifying a subject as having an eye with reduced numbers of corneal nerves as compared to a reference eye, e.g., an eye of a healthy control. In some embodiments of any of the methods described herein, the identifying is performed using in vivo confocal microscopy.

In some embodiments of any of the methods described herein, the administering results in treatment of Fuchs' endothelial corneal dystrophy, pseudophakic bullous keratopathy, pseudoexfoliation syndrome, herpetic stromal keratitis, endothelial cell loss after full-thickness or partial-thickness corneal transplantation, herpes zoster ophthalmicus, uveitis, or graft rejection in the subject. In some embodiments of any of the methods described herein, the subject is administered two or more doses of VIP. In some embodiments of any of the methods described herein, the two or more doses are administered to the subject at a frequency of at least once a month. In some embodiment of any of the methods described herein, the two or more doses are administered to the subject at a frequency of at least once every two weeks. In some embodiments of any of the methods described herein, the two or more doses are administered to the subject at a frequency of at least once every week.

Also provided herein is a VIP or a nucleic acid encoding a VIP for use in reducing nerve loss-related corneal endothelial cell loss in a subject (e.g., a subject identified as having an eye with reduced numbers of corneal nerves as compared to a reference eye, e.g., an eye of a healthy control).

Also provided herein are methods of using a VIP or a nucleic acid encoding a VIP in the manufacture of a medicament for reducing nerve loss-related corneal endothelial cell loss in a subject (e.g., a subject identified as having an eye with reduced numbers of corneal nerves as compared to a reference eye, e.g., an eye of a healthy control).

By the term “nerve loss-related corneal endothelial cell loss” is meant corneal endothelial cell death (e.g., apoptotic cell death or other type of cell death) mediated by (directly or indirectly), associated with, or caused by a neuron loss (neuron death) and/or nerve damage in one or both eyes of a subject. Non-limiting examples of diseases that are characterized by neuron loss and/or nerve damage are described herein. Additional examples of causes of neuron loss and/or nerve damage are known in the art.

By the term “treating” or “efficacy of treatment” is meant a reduction in the number of symptoms of a disease or disorder in a subject (e.g., reduce the number of symptoms of Fuchs' endothelial corneal dystrophy, pseudophakic bullous keratopathy, keratoconus, pseudoexfoliation syndrome, atopic keratoconjunctivitis, herpetic keratitis, endothelial cell loss after full-thickness or partial-thickness corneal transplantation, herpes zoster ophthalmicus, uveitis, or graft rejection), a decrease (e.g., a significant, detectable, or observable decrease) the severity, frequency, and/or duration of one or more (e.g., at least two, three, or four) symptoms of a disease or disorder in a subject (e.g., reduce the severity, frequency, and/or duration of one or more symptoms of Fuchs' endothelial corneal dystrophy, pseudophakic bullous keratopathy, keratoconus, pseudoexfoliation syndrome, atopic keratoconjunctivitis, herpetic keratitis, endothelial cell loss after full-thickness or partial-thickness corneal transplantation, herpes zoster ophthalmicus, uveitis, or graft rejection in a subject), and/or a decrease corneal endothelial cell loss (e.g., nerve loss-related corneal endothelial cell loss) in a subject.

By the term “center of the cornea” or “central cornea” is meant an approximately circular area having a diameter of less than 5 mm (e.g., a diameter less than 4.5 mm, a diameter less than 4 mm, or a diameter of less than 3 mm) from the geometric center point of the cornea.

By the term “peripheral cornea” is meant an area in the cornea that falls outside the center of the cornea (as described above).

By the term “in vivo confocal microscopy” is meant the use of a confocal microscope to visualize one or more tissue(s) (e.g., cornea), cells (e.g., endothelial cells and nerves present in the cornea), and/or cellular substructures (e.g., nerve branching in the cornea) present within a mammal (e.g., a human). Methods of performing in vivo confocal microscopy are described herein.

By the term “length of a nerve” or “nerve length” is generally meant the distance between the cell body (soma) of the nerve cell and the distal end of the axon (end of the axon that is not proximal to the cell body) of the nerve cell, or the distance between (i) a distal end of a dendrite (end of a dendrite that is not proximal to the cell body) that extends from the cell body at a position approximately opposite to the position in the cell body where the axon extends from the cell body, and (ii) the distal end of the axon of the nerve cell. In some embodiments, the length of a nerve or nerve length can be determined in the cornea of a subject using in vivo confocal microscopy methods, e.g., methods known by those skilled in the art or any of the methods described herein. In some embodiments, nerve length is determined, e.g., by in vivo confocal microscopy, and represented as the sum of the length of the nerve fibers observed per frame, and may be converted into units of microns per mm².

By the term “reference value” is meant a value that is used for comparative purposes. In some embodiments, a reference value represents the number of nerves in the eye (e.g., cornea) of a healthy subject (e.g., a subject that does not have an eye disease, e.g., does not present with one or more symptoms of an eye disorder or a subject that has not been diagnosed and/or identified as having an eye disorder). Additional examples of reference values are described herein.

By the term “healthy control” is meant a subject that does not have eye disease. For example, a healthy subject does not present with one or more symptoms of an eye disorder and/or has not been identified or diagnosed as having an eye disorder. For example, a healthy subject as described herein, has also not been exposed to a nerve-damaging agent or stimulus.

By the term “topical solution” as used in herein is meant a pharmaceutically acceptable solution (e.g., buffer) that contains a therapeutically effective amount of one or more (e.g., at least two, three, or four) agents (e.g., VIP).

By the term “subject” is meant any mammal (e.g., a human, mice, rat, and rabbit) who has, or is at risk of developing corneal endothelial cell loss (e.g., nerve loss-related corneal endothelial cell loss).

Other definitions appear in context throughout this disclosure. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1 is a set of four representative images of nerves in the central and peripheral cornea of control mice and mice following trigeminal nerve axotomy.

FIG. 2 is a graph of the nerve density over time in the central and peripheral cornea in control mice (normal) and in mice at 1 week and 2 weeks following axotomy (day 7 and day 14, respectively).

FIG. 3 is a set of eight confocal images of central (top row) and peripheral (bottom row) corneal endothelium in normal and trigeminal nerve axotomized mice.

FIG. 4 is a graph of the number of corneal endothelium cells/100 mm² area over time in the central or peripheral cornea determined in normal (control) mice and trigeminal nerve axotomized mice. The data are represented as a mean±standard error of 3 normal and 3 axotomized corneas per time point.

FIG. 5 is a set of four stacked corneal optical coherence tomography images of a cornea in a control mouse (top image) and cornea from trigeminal nerve axotomized mice over time (bottom three images).

FIG. 6 is a graph of the corneal thickness in control mice, sham-treated mice, and trigeminal nerve axotomized-mice at different time points.

FIG. 7 is a graph of the relative mRNA VIP level in the cornea from trigeminal nerve axotomized-mice at day 3 and day 7, as compared to the mRNA VIP level in control mouse corneas (p<0.05).

FIG. 8 is a graph of the VIP levels in control mouse corneas and the corneas of trigeminal nerve axotomized-mice at day 7 and day 14 (p<0.05).

FIG. 9A is a set of six images from the central and peripheral cornea of trigeminal nerve axotomized-mice treated with PBS injection. Anti-ZO-1 (green) was used to stain the tight junctions of the cells and TORPO-3 (blue) was used to stain the nuclei. The images of negative controls incubated only with the secondary antibody are shown in the left column. The original magnification was 400× with 2× zoom.

FIG. 9B is a set of six images from the central and peripheral cornea of trigeminal nerve axotomized-mice treated with VIP injection. Anti-ZO-1 (green) was used to stain the tight junctions of the cells and TORPO-3 (blue) was used to stain the nuclei. The images of negative controls incubated only with the secondary antibody are shown in the left column. The original magnification was 400× with 2× zoom.

FIG. 10 is a graph of the average number of endothelial cells per 100 mm² area of the central and peripheral cornea of normal mice and trigeminal nerve axotomized-mice at day 7 and day 14 (p<0.05).

FIG. 11 is a graph of the average number of endothelial cells per 100 mm² area of the central and peripheral cornea of normal mice and trigeminal nerve axotomized-mice administered VIP daily. The data are represented as the mean±standard error of three axotomized corneas per time point.

FIG. 12 is a set of three cornea images determined using AS-COT in control mice (top image), trigeminal nerve axotomized-mice administered VIP daily (middle image), and trigeminal nerve axotomized-mice administered phosphate buffered saline (PBS) daily (bottom image).

FIG. 13 is a graph of the corneal thickness in control mice, trigeminal nerve axotomized-mice administered VIP daily (VIP), and trigeminal nerve axotomized-mice administered PBS daily (PBS) (p<0.02).

### DETAILED DESCRIPTION OF THE INVENTION

The invention is based, at least in part, on the discovery that administration of VIP to a mammal decreases corneal endothelial cell loss (e.g., nerve loss-related corneal endothelial cell loss). In view of this discovery, provided herein are methods of decreasing or preventing corneal endothelial cell loss (e.g., nerve loss-related corneal endothelial cell loss) in a mammal that include administering VIP or a nucleic acid encoding VIP to a mammal identified as having a decreased number of nerves in one or both eyes as compared to a healthy control. Also provided are compositions that contain VIP (e.g., VIP or a nucleic acid encoding VIP) and one or more additional agents for treating Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection.

### Vasoactive Intestinal Peptide

Vasoactive intestinal peptide (VIP) is a peptide hormone that is a member of the glucagon/secretin superfamily. VIP is a pre-pro-protein that is processed by proteases of the cell to generate the mature protein. The first isoform of the pre-pro-human VIP is shown below (SEQ ID NO: 1). The mature sequence of the first isoform of pre-pro-VIP is underlined and in bold below (SEQ ID NO: 2). The signal peptide is amino acids 1-25 of SEQ ID NO: 1, and the pro-protein (prior to the last proteolytic cleavage events to generate the mature protein) is amino acids 26-170 of SEQ ID NO: 1. The cDNA sequence encoding the first isoform of pre-pro-human VIP is shown below (SEQ ID NO: 3)

| First Isoform Pre-Pro-Human VIP | |
|---|---|
| (SEQ ID NO: 1) | |
| 1 | mdtrnkaqll vlltllsvlf sqtsawplyr apsalrlgdr ipfeganepd qvslkedidm |
| 61 | lqnalaendt pyydvsrnar hadgvftsdf skllgqlsak kyleslmgkr vssnisedpv |
| 121 | pvkrhsdavf tdnytrlrkq mavkkylnsi lngkrssege spdfpeelek |
| cDNA encoding First Isoform of Pre-Pro-Human VIP | |
| (SEQ ID NO: 3) | |
| 1 | agggtagagt gagaagcacc agcaggcagt aacagccaac ccttagccat tgctaagggc |
| 61 | agagaactgg tggagccttt ctcttactcc caggacttca gcacctaaga cagctccaaa |
| 121 | acaaaccaga acagtcagct ccgggggagc acgactgggc gagaggcaca gaaatggaca |
| 181 | ccagaaataa ggcccagctc cttgtgctcc tgactcttct cagtgtgctc ttctcacaga |
| 241 | cttcggcatg gcctctttac agggcacctt ctgctctcag gttgggtgac agaataccct |
| 301 | ttgagggagc aaatgaacct gatcaagttt cattaaaaga agacattgac atgttgcaaa |
| 361 | atgcattagc tgaaaatgac acaccctatt atgatgtatc cagaaatgcc aggcatgctg |
| 421 | atggagtttt caccagtgac ttcagtaaac tcttgggtca actttctgcc aaaaagtacc |
| 481 | ttgagtctct tatgggaaaa cgtgttagca gtaacatctc agaagaccct gtaccagtca |
| 541 | aacgtcactc agatgcagtc ttcactgaca actatacccg ccttagaaaa caaatggctg |
| 601 | taaagaaata tttgaactca attctgaatg gaaagaggag cagtgaggga gaatctcccg |
| 661 | actttccaga agagttagaa aaatgatgaa aaagaccttt ggagcaaagc tgatgacaac |
| 721 | ttcccagtga attcttgaag gaaaatgata cgcaacataa ttaaattttg agttctacat |
| 781 | aagtaattca agaaaacaac ttcaatatcc aaaccaaata aaaatattgt gttgtgaatg |
| 841 | ttgtgatgta ttctagctaa tgtaataact gtgaagttta cattgtaaat agtatttgag |
| 901 | agttctaaat tttgtcttta actcataaaa agcctgcaat ttcatatgct gtatatcctt |
| 961 | tctaacaaaa aaatatattt aatgataagt aaatgctagg ttaattccaa ttatatgaga |
| 1021 | cgtttttgga agagtagtaa tagagcaaaa ttgatgtgtt tatttataga gtgtacttaa |
| 1081 | ctattcagga gagtagaaca gataatcagt gtgtctaaat ttgaatgtta agcagatgga |
| 1141 | atgctgtgtt aaataaacct caaaatgtct aagatagtaa caatgaagat aaaaagacat |
| 1201 | tcttccaaaa agattttcag aaaatattat gtgtttccat attttatagg caacctttat |
| 1261 | ttttaatggt gttttaaaaa atctcaaatt tggattgcta atcaccaaag gctctctcct |
| 1321 | gatagtcttt cagttaagga gaacgacccc tgcttctgac actgaaactt ccctttctgc |
| 1381 | ttgtgttaag tatgtgtaaa atgtgaagtg aatgaaacac tcagttgttc aataataaat |
| 1441 | atttttgcca taatgactca gaatattgct ttggtcatat gagcttcctt ctgtgaaagt |
| 1501 | acatttggag acacaactat ttttccaaaa taattttaag aaatcaaaga gagaaaataa |
| 1561 | agaccttgct tatgattgca gataaaaaaa aaaaaaaaaa a |

The second isoform of the pre-pro-human VIP is shown below (SEQ ID NO: 4). The mature sequence of the second isoform of pre-pro-human VIP is underlined (SEQ ID NO: 5). The signal peptide is amino acids 1-25 of SEQ ID NO: 4, and the pro-protein (prior to the last proteolytic cleavage event to generate the mature protein) is amino acids 26-169 of SEQ ID NO: 4. The cDNA sequence encoding the second isoform of pre-pro-human VIP is shown below (SEQ ID NO: 6).

| Second Isoform Pre-Pro-Human VIP | |
|---|---|
| (SEQ ID NO: 4) | |
| 1 | mdtrnkaqll vlltllsvlf sqtsawplyr apsalrlgdr ipfeganepd qvslkedidm |
| 61 | lqnalaendt pyydvsrnar hadgvftsdf skllgqlsak kyleslmgkr vsnisedpvp |
| 121 | vkrhsdavft dnytrlrkqm avkkylnsil ngkrsseges pdfpeelek |
| cDNA encoding Second Isoform of Pre-Pro-Human VIP | |
| (SEQ ID NO: 6) | |
| 1 | agggtagagt gagaagcacc agcaggcagt aacagccaac ccttagccat tgctaagggc |
| 61 | agagaactgg tggagccttt ctcttactcc caggacttca gcacctaaga cagctccaaa |
| 121 | acaaaccaga acagtcagct ccgggggagc acgactgggc gagaggcaca gaaatggaca |
| 181 | ccagaaataa ggcccagctc cttgtgctcc tgactcttct cagtgtgctc ttctcacaga |
| 241 | cttcggcatg gcctctttac agggcacctt ctgctctcag gttgggtgac agaataccct |
| 301 | ttgagggagc aaatgaacct gatcaagttt cattaaaaga agacattgac atgttgcaaa |
| 361 | atgcattagc tgaaaatgac acaccctatt atgatgtatc cagaaatgcc aggcatgctg |
| 421 | atggagtttt caccagtgac ttcagtaaac tcttgggtca actttctgcc aaaaagtacc |
| 481 | ttgagtctct tatgggaaaa cgtgttagta acatctcaga agaccctgta ccagtcaaac |
| 541 | gtcactcaga tgcagtcttc actgacaact atacccgcct tagaaaacaa atggctgtaa |
| 601 | agaaatattt gaactcaatt ctgaatggaa agaggagcag tgagggagaa tctcccgact |
| 661 | ttccagaaga gttagaaaaa tgatgaaaaa gacctttgga gcaaagctga tgacaacttc |
| 721 | ccagtgaatt cttgaaggaa aatgatacgc aacataatta aattttgagt tctacataag |
| 781 | taattcaaga aaacaacttc aatatccaaa ccaaataaaa atattgtgtt gtgaatgttg |
| 841 | tgatgtattc tagctaatgt aataactgtg aagtttacat tgtaaatagt atttgagagt |
| 901 | tctaaatttt gtctttaact cataaaaagc ctgcaatttc atatgctgta tatcctttct |
| 961 | aacaaaaaaa tatatttaat gataagtaaa tgctaggtta attccaatta tatgagacgt |
| 1021 | ttttggaaga gtagtaatag agcaaaattg atgtgtttat ttatagagtg tacttaacta |
| 1081 | ttcaggagag tagaacagat aatcagtgtg tctaaatttg aatgttaagc agatggaatg |
| 1141 | ctgtgttaaa taaacctcaa aatgtctaag atagtaacaa tgaagataaa aagacattct |
| 1201 | tccaaaaaga ttttcagaaa atattatgtg tttccatatt ttataggcaa cctttatttt |
| 1261 | taatggtgtt ttaaaaaatc tcaaatttgg attgctaatc accaaaggct ctctcctgat |
| 1321 | agtctttcag ttaaggagaa cgacccctgc ttctgacact gaaacttccc tttctgcttg |
| 1381 | tgttaagtat gtgtaaaatg tgaagtgaat gaaacactca gttgttcaat aataaatatt |
| 1441 | tttgccataa tgactcagaa tattgctttg gtcatatgag cttccttctg tgaaagtaca |
| 1501 | tttggagaca caactatttt tccaaaataa ttttaagaaa tcaaagagag aaaataaaga |
| 1561 | ccttgcttat gattgcagat aaaaaaaaaa aaaaaaaa |

A human VIP administered to a subject in any of the methods described herein can consist of a sequence of either SEQ ID NO: 2 or 4, or can contain a sequence of either SEQ ID NO: 2 or 4 (and optionally, contain no more than 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acid residues). A human VIP administered to a subject in any of the methods described herein can consist of a sequence of either SEQ ID NO: 2 or 4 with one, two, or three amino acid substitutions (e.g., conservative substitutions), insertions, deletions, or additions. As one skilled in the art can appreciate, the amino acids conserved among different mammalian species (e.g., conserved among the human, mouse, and rat mature, pre-pro, or pro-VIPs) should not be substituted or deleted, while amino acid positions that are different among different mammalian species should be substituted or deleted. A description of the mouse and rat VIPs is provided below. A VIP administered to the subject in any of the methods described herein can also be any wildtype mammalian mature VIP (e.g., any wildtype mature human VIP).

In any of the methods described herein, a nucleic acid (e.g., an expression vector) encoding VIP can be administered to the subject. Non-limiting examples of cDNAs that encode human VIP are SEQ ID NO: 3 and SEQ ID NO: 6. A nucleic acid encoding VIP that can be administered to a subject can contain a sequence of SEQ ID NO: 3 or SEQ ID NO: 6, or can contain a sequence that is at least 90%, 91%, 92%, 93%, 94%, 95%, 96%, 97%, 98%, 99%, of 100% identical to SEQ ID NO: 3 to SEQ ID NO: 6. In some examples, a nucleic acid encoding VIP contains nucleotides 546 to 629 of SEQ ID NO: 3 or nucleotides 543 to 626 of SEQ ID NO: 6. In other examples, a nucleic acid encoding VIP contains a sequence that is at least 90%, 91%, 92%, 93%, 94%, 95%, 96%, 97%, 98%, 99%, or 100% identical to nucleotides 546 to 629 of SEQ ID NO: 3 or nucleotides 543 to 626 of SEQ ID NO: 6. A nucleic acid administered to a subject in the methods described herein can encode any of VIP described herein (e.g., any of the VIPs described in the above paragraph). A nucleic acid encoding a VIP can be an expression vector (e.g., naked DNA, a lentivirus vector, an adenovirus vector, or a retroviral vector). The expression vector can contain a sequence encoding a VIP operably linked to a heterologous promoter (e.g., a hamster rpS21 promoter, hamster β-actin promoter, and SV40 early promoter). Exemplary expression vectors and methods for administering expression vectors to the eye are described in Banin et al., Invest. Ophthalmol. Vis. Sci. 44:1529-1533, 2003).

The isoform of the pre-pro-mouse VIP is shown below (SEQ ID NO: 7). The mature sequence of mouse VIP is underlined and in bold (SEQ ID NO: 8). The signal peptide is amino acids 1-25 of SEQ ID NO: 7, and the pro-protein (prior to the last proteolytic cleavage event to generate the mature protein) is amino acids 26-169 of SEQ ID NO: 7. The cDNA sequence encoding mouse pre-pro-VIP is shown below (SEQ ID NO: 9).

| Pre-Pro-MouseVIP | |
|---|---|
| (SEQ ID NO: 7) | |
| 1 | mearskpqfl aflilfsvlf sqslawplfg ppsvvsrldd rmpfegagdp dqvslkadsd |
| 61 | ilqnplaeng tpyydvsrna rhadgvftsd ysrllgqisa kkylesligk risssisedp |
| 121 | vpikrhsdav ftdnytrlrk qmavkkylns ilngkrsseg dsadfleele k |
| cDNA encoding Pre-Pro-Mouse VIP | |
| (SEQ ID NO: 9) | |
| 1 | agcttggaca gcagagcact agccagctac agccaaccgt tccccaggaa ccgggaacag |
| 61 | actggtggag ccttccctag agcagaactt cagcacccta gacagctgcc acgaagccgg |
| 121 | aaaggcagcc ctgcctgaag gaaacagcca aggaggcacc gagatggaag ccagaagcaa |
| 181 | gcctcagttc ctggcattcc tgatactctt cagtgtgctg ttctctcagt cgctggcctg |
| 241 | gcctctcttt ggaccacctt ctgtagtgag taggctggat gacaggatgc cgtttgaagg |
| 301 | agcaggtgac cctgaccaag tctctttaaa agcagactct gacatcttgc agaatccctt |
| 361 | agcagaaaat ggcacaccct attatgatgt gtcaagaaat gccaggcatg ctgatggagt |
| 421 | tttcaccagc gattacagca gacttctggg tcagatttct gccaaaaaat accttgagtc |
| 481 | actcattggc aaacgaatca gcagcagcat ctcggaagat cctgtgccaa tcaaacgaca |
| 541 | ctctgatgcc gtcttcacag ataactacac ccgcctcaga aagcaaatgg ctgtgaagaa |
| 601 | atacctgaac tccatcctga atggaaagag gagcagtgag ggagattctg cagactttct |
| 661 | tgaagagctg gagaaatgat gggaagaggc ctctgggcag agctgaaatc agagaattct |
| 721 | cgaaggaaaa caaccacgtg attacattat gagttctaca tgtctaattc aagaaaaaaa |
| 781 | cttccatagc aaaaccaaat aaaatgtgtt gtgaatattg tggtttcctt tatgtaataa |
| 841 | ctgtgatgtt tacattgtaa atattatttg agcattctaa cattcatctg tagctcatga |
| 901 | aatgcttata atttcatatg ctatatattc tttcaaagaa aagtatattt aatgataggt |
| 961 | agatactaga ttaattgcaa ttatctgaag ctttctgcaa gggtagcaat cgaggaaaat |
| 1021 | tgatgtgttt atttatagca tgtagttaac tattcaacag agcagaacag ataatcagtg |
| 1081 | tgaacaagtc taaatgctaa gcagataggc tgctgtgtta cataaggcaa aatatctaag |
| 1141 | gggaataaca aattatggat aaaagagata tgtggcaaaa ggattttcag aattgtattt |
| 1201 | ctccagtgat aggtactcca tctctcacgg attcatctct cccattaggc tttgcaatcc |
| 1261 | ccaaaggcta cttcagagat gcttcagcta ggaaaagccc atcgtccaat ctggggcttc |
| 1321 | ccctttctgc gtgtgctatg gatgtgtaaa ctagaagcta aatggagtgc ttgatttcca |
| 1381 | gtagtaaata cttctcccat agtcactcac aatgatattt tgtcttattg gcttcctttg |
| 1441 | ctgaaagtac atttgtagac acaactattt ttccaatgtg attgtatgaa attaaagaca |
| 1501 | ggaataaaga tctttggtta tcattgc |

The isoform of the pre-pro-rat VIP is shown below (SEQ ID NO: 10). The mature sequence of the isoform of rat VIP is underlined (SEQ ID NO: 11). The signal peptide is amino acids 1-25 of SEQ ID NO: 10, and the pro-protein (prior to the last proteolytic cleavage event to generate the mature protein) is amino acids 26-170 of SEQ ID NO: 10. The cDNA sequence encoding the pre-pro-rat VIP is shown below (SEQ ID NO: 12).

| Pre-Pro-Rat VIP | |
|---|---|
| (SEQ ID NO: 10) | |
| 1 | mesrskpqfl ailtlfsvlf sqslawplyg ppssvrlddr lqfegagdpd qvslkadsdi |
| 61 | lqnalaendt pyydvsrnar hadgvftsdy srllgqisak kylesligkr isssisedpv |
| 121 | pvkrhsdavf tdnytrlrkq mavkkylnsi lngkrssegd spdfleelek |
| cDNA encoding Pre-Pro-Rat VIP | |
| (SEQ ID NO: 12) | |
| 1 | ctagcggcta ctgccaacct ttccccagga ccaggggcag actccgtgga gccttctccc |
| 61 | aagcagaact tcagcacccc agacagctcc cccgcgccgg agagacggtc ctgccagaag |
| 121 | gaaagaccca aggaggcacc gagatggaat ccagaagcaa gcctcagttc ctggcgatcc |
| 181 | tgacactctt cagtgtgctg ttctcacagt cgctggcctg gcctctctat gggccacctt |
| 241 | cttcagtgag gttggatgac aggctgcagt tcgaaggagc aggtgaccct gatcaagtct |
| 301 | ctttaaaagc agactctgac atcttgcaga atgccttagc ggagaatgac acgccctatt |
| 361 | atgatgtgtc cagaaatgcc aggcatgctg atggagtttt caccagcgac tacagtagac |
| 421 | ttctgggtca gatttctgcc aaaaaatacc ttgagtcact cattggcaaa cgaatcagca |
| 481 | gtagcatctc ggaagacccc gtgccggtca aacgacactc tgatgcagtc ttcacagata |
| 541 | actacacccg ccttagaaag caaatggctg tgaagaaata cttgaactcc attctaaatg |
| 601 | ggaagaggag cagtgaggga gattctccag acttccttga agagctagag aaatgatgag |
| 661 | aagggtcctc tgggcagagc tgaagatcag agaattcttg aaggaaaaca accaagtgat |
| 721 | tacattatga gttctacata tctaattcaa gaaaacaact tccatagcaa aaccaaataa |
| 781 | aatgtgttgt gaatattgtg gtttccttta tgtaataact gtgatgttta cattgtaaat |
| 841 | atatttagca ctctaaaatt catctttagc tcgtgaaagg cttataattt catatgctat |
| 901 | atattcttta aaaaatatat ttaatgatag gtagatacta gattaattgc aattatctga |
| 961 | agctttctgc aagggtagca atcgaggaaa attgatgggc ttatttatag catgcagtta |
| 1021 | actattcaac agagcagaac agataatcag tgtgaccaag tctgaatgct aagcagatag |
| 1081 | gctgccgtgt tacataaagc aaaatatcta agggaaaacc aaacatatgg aaaatggaga |
| 1141 | tacttgacaa aaggattttc aaaattgtat tcctccagtg atagggactc cacctctcat |
| 1201 | ggattcatct ctccgactag gatttgcaat ccccaaaagc ttcttcgagt tgcttcagct |
| 1261 | aggaaaagct caacttccaa cctggagctt ccccttcctg cttgtgctgt ggatgtgtaa |
| 1321 | gctagaagcc taacggagtg cttgatttcc agtagtaaat actctttccg taatcactca |
| 1381 | caacagtatt ttgtcttatt ggcttccttt gctgaaagta catttgtaga cacaactatt |
| 1441 | tttccaatgt gattgtatga aattaaagac aggaataaag atctttggtt atcattgcaa |
| 1501 | aaaaaaaaaa aaaaaaaaaa aaaaaaaaaa aa |

In some embodiments of any of the methods or compositions described herein, the VIP contains at least one (e.g., two, three, four, five, or six) modifications (e.g., the N-terminus can be acetylated, the C-terminus can be amidated, the VIP can contain at least one D-amino acid, the VIP can contain at least one non-natural amino acid, and the VIP can be conjugated to a stabilizing moiety). Non-limiting examples of stabilizing moieties that can be conjugated to a VIP include a lipid (e.g., myristic acid, palmitic acid, or stearic acid), a protein (e.g., serum albumin or an Fc region of an antibody), or a polymer (e.g., a polyethylene glycol or poly(lactide-co-glycolide)). In some embodiments, a VIP can contain at least one (e.g., two, three, four, five, six, seven, eight, nine, ten, eleven, twelve, thirteen, fourteen, fifteen, sixteen, seventeen, eighteen, nineteen, or twenty) D-amino acids, or all of the amino acids in VIP can be D-amino acids. In some embodiments, a VIP protein can contain at least one non-natural amino acid (e.g., citrulline, ornithine, ε-acetyl-lysine, β-alanine, aminobenzoic acid, 6-aminocaprioc acid, aminobutyric acid, acetamidomethyl protected cysteine, dimethyl-lysine, hydroxyl-proline, mercaptopropionic acid, methyl-lysine, 3-nitro-tyrosine, norleucine, pyro-glutamic acid, and carbobenzoxyl).

In some embodiments, a nucleic acid encoding VIP can contain at least one modified nucleotide (e.g., modified in a base and/or in the sugar) and/or at least one modification of a phosphodiester bond. Non-limiting examples of modified nucleotides include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. In some embodiments, the at least one modification of a phosphodiester bond is a phosphorothioate bond or a psuedopeptide backbone (see, Hyrup et al., Bioorg. Med. Chem. 4:5-23, 1996). In some embodiments, a subject can be administered a nucleic acid containing a sequence encoding a VIP that is operably linked to a heterologous promoter.

### Diseases Characterized by Corneal Endothelial Cell Loss

A variety of eye diseases, including non-inflammatory eye diseases (e.g., endothelial corneal dystrophy, keratoconus, and pseudoexfoliation syndrome) and inflammatory eye diseases (e.g., atopic keratoconjunctivitis and herpetic stromal keratitis), are characterized by corneal endothelial cell loss (e.g., nerve loss-related corneal endothelial cell loss). Corneal endothelial cell loss (e.g., nerve loss-related endothelial cell loss) can also occur after full-thickness or partial-thickness corneal transplantation or be caused by herpes zoster ophthalmicus, uveitis, pseudophakic bullous keratopathy, or graft rejection.

Non-limiting symptoms of Fuchs' endothelial corneal dystrophy include blurred vision on awakening that may gradually clear up as the day goes on, distorted vision, sensitivity to light, difficulty seeing at night and seeing halos around lights, eye discomfort, epithelial blisters on the surface of the cornea, cloudy or hazy cornea, and decreased numbers of corneal nerves.

Non-limiting symptoms of keratoconus include blurred or distorted vision, increased sensitivity to bright light or glare, problems with night vision, many changes in eyeglass prescriptions, the sudden worsening or clouding of vision caused by a condition in which the back of the cornea fills with fluid (hydrops), and enlarged or decreased numbers of corneal nerves.

Non-limiting symptoms of pseudoexfoliation syndrome include lessened visual activity, changes in perceived visual field, microscopic white or grey granular flakes within the eye (e.g., ocular and extraocular flakes), decreased subbasal nerve density, and increased tortuosity of the corneal nerves.

Non-limiting symptoms of atopic keratoconjunctivitis include blurry vision, eye pain, eye redness, eye irritation, eye discharge, hazy or cloudy cornea, photophobia, increased tearing, conjunctival swelling, eyelid swelling, and enlarged or decreased numbers corneal nerves.

Non-limiting symptoms of herpetic stromal keratitis include pain, photophobia, lacrimation, blepharospasm, reduced vision, and reduced numbers of corneal nerves.

Non-limiting symptoms of corneal endothelial cell loss after full-thickness or partial-thickness corneal transplantation include eye redness, eye pain, irritated eyes, light-sensitive eyes, impaired vision, and reduced numbers of corneal nerves.

Non-limiting symptoms of graft rejection include pain at the site of the transplant, feeling unwell, crankiness, flu-like symptoms, fever, weight changes, swelling, change in heart fate, and urinating less often.

Non-limiting symptoms of uveitis include eye redness, eye pain, light sensitivity, blurred vision, dark, floating spots in field of vision, decreased vision, and hypopyon.

Non-limiting symptoms of herpes zoster ophthalmicus include red eye, eye irritation, reduced visual acuity, eye pain, eye tearing, light sensitivity, and corneal inflammation.

Non-limiting symptoms of pseudophakic bullous keratopathy include poor vision, discomfort, pain, and stromal edema.

Subjects can be diagnosed as having Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection by a medical professional (e.g., a physician, a physician's assistant, a nurse, a nurse's assistant, or a laboratory technician). A subject diagnosed as having Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection may present with one or more (e.g., two, three, four, five, six, seven, eight, nine, and ten) of the symptoms of Fuchs' endothelial corneal dystrophy, pseudophakic bullous keratopathy, keratoconus, pseudoexfoliation syndrome, atopic ketaroconjunctivitis, herpetic stromal keratitis, endothelial cell loss after full-thickness or partial-thickness corneal transplantation, herpes zoster ophthalmicus, uveitis, or graft rejection, respectively, described herein. In some embodiments, the subject may be receiving a treatment or have previously received a treatment for Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection. In some examples, the subject may have been in contact with a nerve-damaging agent or stimulus (e.g., any of the exemplary nerve-damaging agents or stimuli described herein).

### Methods of Detecting Corneal Nerves and Corneal Endothelial Cells

In vivo microscopy (e.g., in vivo confocal microscopy) is a noninvasive procedure that allows the imaging of the living cornea at the cellular level. Additional non-invasive procedures can be used to perform the methods described herein. A non-invasive procedure, e.g., is one that does not require the puncturing or incision in the tissue of the subject (e.g., in the cornea of the subject).

In vivo microscopy is a technique that enables the study of corneal endothelial cells and corneal corneal nerves (e.g., those present in the central or peripheral cornea of a subject). Exemplary methods for detecting these specific cells are described herein.

In vivo confocal microscopes are commercially available from, e.g., Nidek Technologies (Gamagori, Japan) and Heidelberg Engineering GmbH (Dossenheim, Germany). In the methods described herein, the confocal microscopes are commonly equipped with a 35× to 70× immersion lens. For example, a Confoscan microscopy equipped with a 40×/0.75 objective lens or a Heidelberg Engineering GmbH microscope can be equipped with a 63× water-contact lens covered with a sterile single-use polymethylmethacrylate cap (Tomocap, Heidelberg Engineering). The Confoscan microscope, e.g., can produce images of 460 μm by 345 μm, with a magnification of 500× and a lateral resolution of 1 μm/pixel. The Heidelberg microscope, e.g., can produce images of 400 μm by 400 μm, with a magnification of 800× and a lateral resolution of 1 mm/pixel. The subject is typically administered a topical anesthesia (e.g., 0.5% proparacaine hydrochloride) prior to contacting the immersion lens with the subject's eye tissue. A subject can also be administered a lubricating solution (e.g., 2.5% hydroxypropyl methylcellulose) prior to contacting the immersion lens with the subject's eye tissue. The digital images collected can be stored on a computer workstation using commonly known methods. The resulting images can be analyzed using a variety of commercially available software. Non-limiting examples of software that can be used to analyze the collected images include ImageJ software (ImageJ software described in Girish et al., Indian J. Cancer 41:47, 2004) and NeuronJ software (Meijering et al., Cytometry A 58:167-176, 2004).

Changes in the density or average length of nerves present in the cornea, in the amount of branching in nerves present in the cornea, and in the total number or density of nerves present in the cornea can be determined using confocal microscopy. Exemplary in vivo confocal microscopic methods for determining the change in corneal nerve cell density are described in the Example. However, the methods described in the Example are not limiting. One skilled in the art will recognize that modifications of these methods can be made (e.g., change in the level of magnification) without significantly compromising the quality of the images obtained.

Nerve analysis can be done, e.g., using a software program (e.g., the semi-automated tracing program NeuronJ (Meijering et al., Cytometry A 58:167-176, 2004), a plug-in for ImageJ (ImageJ software described in Girish et al., Indian J. Cancer 41:47, 2004)). In some embodiments, nerve density can be assessed by measuring the total length of the nerve fibers in micrometers per frame. Nerve branching is defined as the total number of nerve branches in one image. The number of total nerves measured is defined as the number of all nerves, including main nerve trunks and branches in one image. Although exemplary software programs are recited above, skilled artisans will appreciate that a number of other suitable software programs are available. The number of corneal nerves in a subject can be determined using any of the exemplary methods described herein or known in the art. Additional methods for assessing nerve damage or nerve loss in the cornea are described in U.S. Patent Application Ser. No. 61/601,149, filed Feb. 21, 2012, and International Patent No. PCT/US2013/027181, filed Feb. 21, 2013.

The efficacy of the administration of VIP in reducing corneal endothelial cell loss (e.g., nerve loss-related corneal endothelial cell loss) can be assessed by detecting the numbers and changes in the morphology of corneal endothelial cells in a subject using in vivo confocal microscopy or specular microscopy. Exemplary in vivo confocal microscopic methods for detecting the numbers, density, and changes in the morphology of corneal endothelial cells are described in the Example. However, the methods described in the Example are not limiting. One skilled in the art will recognize that modifications of these methods can be made (e.g., change in the level of magnification, change in autobrightness, the use of gel or the type of caps for the microscope objective lens) without significantly compromising the quality of the images obtained. In some embodiments, two or more images (e.g., three, four, or five images) can be obtained from an eye of the subject. The number, density, and morphological changes in the corneal endothelial cells can be assessed using methods known in the art, e.g., the ImageJ, NIDEK, and Cell Count, Heidelberg Engineering GmbH software. Additional methods for assessing the number, density, and morphological changes in corneal endothelial cells are described in U.S. Patent Application Ser. No. 61/601,149, filed Feb. 21, 2012, and International Patent No. PCT/US2013/027181, filed Feb. 21, 2013.

### Methods of Treating a Subject

Provided herein are methods of reducing corneal endothelial cell loss (e.g., nerve loss-related corneal endothelial cell loss) in a subject (e.g., a human) that include selecting a subject identified as having an eye with reduced numbers of corneal nerves as compared to a reference eye, e.g., an eye of the subject before development of nerve loss, an unaffected eye of the subject, or an eye of a healthy control, and administering VIP or a nucleic acid encoding VIP (e.g., a therapeutically effective amount of VIP or a therapeutically effective amount of a nucleic acid encoding VIP) to the selected subject.

In any of the methods described herein, the subject can present clinically with two or more (two, three, four, or five) symptoms of Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection (e.g., exemplary symptoms of each disorder are described herein). In other examples, the subject does not present clinically with two or more symptoms of Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection (e.g., exemplary symptoms of each disorder are described herein). The subject can be suspected of having Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection. In other examples, the subject has Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection; has been diagnosed as having Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection (e.g., using any of the methods described herein); or has received or is receiving a treatment for Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection. In some embodiments, the subject has been exposed to a nerve-damaging agent or stimulus (e.g., chemotherapy, radiation treatment, drug abuse, heavy metals, pestacides acetylene, atrazine, benzene, ethylene glycol, and mercury). In some embodiments, the subject is administered VIP or a nucleic acid encoding VIP shortly after exposure to a nerve-damaging agent or stimulus (e.g., within 2 weeks, within 1 week, within 6 days, within 5 days, within 4 days, within 3 days, within 2 days, or within 1 day) after exposure). In other examples, the subject is administered a VIP or a nucleic acid encoding VIP shortly after the subject is first identified (e.g., by in vitro confocal microscopy) as having an eye with reduced numbers of corneal nerves as compared to a reference eye, e.g., an eye of a healthy control (e.g., within 2 weeks, within 1 week, within 6 days, within 5 days, within 4 days, within 3 days, within 2 days, or within 1 day) after first identification).

In any of the methods described herein, the corneal endothelial cell loss (e.g., nerve loss-related endothelial cell death) is not caused or mediated (e.g., substantially caused or mediated) by oxidative stress. In some examples, the endothelial cell death is apoptosis.

The subject can be male or female. In any of the methods described herein, the subject can be a child, a teenager, or an adult (e.g., at least 18, 25, 30, 40, 50, 60, 70, 80, or 90 years old).

In some examples, the selecting is performed by retrieving data from the subject's clinical file or analyzing previously obtained in vitro confocal microscopy images obtained from an eye in the subject. Some examples of the present methods further include identifying a subject as having an eye with reduced numbers of corneal nerves as compared to a reference eye, e.g., an eye of a healthy subject or by comparison to a reference value that corresponds to the average number of corneal nerves in the eyes of healthy subjects. The identifying can include performing in vivo confocal microscopy on an eye of a subject (e.g., a subject suspected of having corneal nerve loss or suspected of having Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection, or a subject exposed to a nerve-damaging agent or stimulus).

In some embodiments, these methods are performed by a medical professional (e.g., a physician, a physician's assistant, a nurse, a nurse's assistant, or a laboratory technician). The VIP or nucleic acid encoding VIP can be administered to the subject via intravenous administration, subconjunctival injection, topical administration, oral administration, intramuscular administration, subcutaneous administration, nasal administration, intaarterial administration, intraocular administration, intraorbital administration, or intraperitoneal administration. For example, the VIP or nucleic acid encoding VIP is administered systemically (e.g., by oral or intravenous administration or any other routes of systemic administration described herein or known in the art). In some embodiments, the VIP is formulated as a sustained-release or a deposit formulation (see, e.g., the formulations described in U.S. Pat. No. 5,422,116). In some embodiments, the VIP or the nucleic acid encoding VIP is administered as a nanoparticle (e.g., a biodegradable nanoparticle containing VIP or a nucleic acid encoding VIP). In some embodiments, VIP or nucleic acid encoding VIP is administered by scleral diffusion.

The VIP administered to the subject can be any of the exemplary VIPs described herein. The nucleic acid administered to the subject can be any of the exemplary nucleic acids (e.g., expression vectors) encoding VIP described herein. For example, the VIP can consist of SEQ ID NO: 2 and SEQ ID NO: 5, can comprise a sequence of SEQ ID NO: 2 or SEQ ID NO: 5 and have no more than 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 total amino acid residues. In some examples, the VIP consists of a sequence of SEQ ID NO: 2 or SEQ ID NO: 5, except that one, two, or three amino acid residues are substituted, deleted, inserted, or added. In other examples, the VIP is any wildtype mature human VIP.

In some examples, the subject is administered two or more doses of VIP or a nucleic acid encoding a VIP (e.g., at least 3, 4, 5, 6, 7, 8, 9, 10, 11, 12, 13, 14, 15, 16, 17, 18, 19, or 20 doses). In such examples, the two or more doses can be administered to the subject at a frequency of at least once every six months (e.g., at least once every five months, at least once every four months, at least once every three months, at least once every two months, at least once every month, at least once every three weeks, at least once every two weeks, at least once every week, at least twice a week, at least three times a week, at least four times a week, at least five times a week, at least six times a week, at least once a day, at least twice a day, at least three times a day, or at least four times a day). In some embodiments, the subject continues to receive periodic administration of VIP or a nucleic acid encoding a VIP over a total period of time of greater than 1 year, greater than 2 years, greater than 3 years, greater than 4 years, greater than 5 years, greater than 6 years, greater than 7 years, greater than 8 years, greater than 9 years, greater than 10 years, greater than 15 years, greater than 20 years, greater than 25 years, greater than 30 years, greater than 35 years, greater than 40 years, greater than 45 years, greater than 50 years, greater than 55 years, greater than 60 years, greater than 65 years, or greater than 70 years.

The amount of VIP or nucleic acid encoding VIP in each dose administered to the selected subject can range from about 0.001 to 30 mg/kg body weight, about 0.01 to 25 mg/kg body weight, about 0.1 to 20 mg/kg body weight, about 1 to 10 mg/kg, 2 to 9 mg/kg, 3 to 8 mg/kg, 4 to 7 mg/kg, or 5 to 6 mg/kg body weight.

Some embodiments further include administering one or more agents useful for treating Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection (e.g., any of the exemplary agents described herein or known in the art) to the subject. In such examples, the VIP or the nucleic acid encoding VIP, and the one or more agents useful for treating Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection can be administered at substantially the same time. In other examples, the VIP or the nucleic acid encoding VIP and the one or more agents useful for treating Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection can be administered at different frequencies and/or at different time points to the selected subject. In some embodiments, the subject is administered a composition (e.g., a pharmaceutical composition) that contains both VIP and one or more agents for Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection (e.g., any of the exemplary pharmaceutical compositions described herein).

In some examples, the selected subject has or is diagnosed as having Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection and the administering can result in the treatment of Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection. For example, treatment can result in a reduction in the number of symptoms of Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection in the subject, a reduction (e.g., a significant, detectable, or observable decrease) in the severity, frequency, and/or duration of one or more (e.g., at least two, three, or four) symptoms of Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection in the selected subject, and/or a decrease corneal endothelial cell loss (e.g., nerve loss-related corneal endothelial cell loss) in the selected subject over time.

In some embodiments, the selected subject may already be taking one or more pharmaceutical agents for treatment of Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection (e.g., one or more pharmaceutical agents for treatment of Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection described herein), and the subject is instructed or advised to discontinue taking one or more of the previously prescribed one or more pharmaceutical agents. In some embodiments, the subject may already be taking one or more pharmaceutical agents for treatment of Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection and the VIP or the nucleic acid encoding VIP is administered to the subject in combination with the one or more pharmaceutical agents previously taken by the subject.

The invention is further described in the following example, which does not limit the scope of the invention described in the claims.

### Pharmaceutical Compositions

Also provided are pharmaceutical compositions that contain VIP or a nucleic acid encoding a VIP, and one or more agents useful for treating Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection (e.g., beta-blockers (e.g., levobunolol or timolol), prostaglandin analogues (e.g., latanoprost), a corticosteroid (e.g., dexamethasone, fluorometholone, prednisolone, and rimexolone), an antibiotic, an antiviral agent (e.g., cidofovir), artificial tears, an anti-histamine, trifluridine, an anti-inflammatory non-steroidal drug (NSAID) (e.g., diclofenac and ketorolac), a cycloplegic (e.g., atropine, cyclopentolate, homatropine, scopolamine, and tropicamide, or any combination thereof).

In some embodiments, the one or more agents for treating Fuchs' endothelial corneal dystrophy, pseudophakic bullous keratopathy, keratocoma, pseudoexfoliation syndrome, atopic keratoconjunctivitis, herpetic stromal keratitis, endothelial loss after full-thickness or partial-thickness corneal transplantation, herpes zoster ophthalmicus, uveitis, or graft rejection is a non-natural agent or a non-naturally occurring agent.

In some embodiments, the compositions are formulated with a pharmaceutically acceptable carrier. The pharmaceutical compositions and formulations can be administered intraperitoneally, intravenously, intranmuscularly, subconjunctivally, transdermally, nasally, intraorbitally, parenterally, or orally, or by local, topical, administration, such as by eye drops or local injection, or by scleral diffusion. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The pharmaceutical compositions provided herein may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring, and perfuming agents, preservatives, and antioxidants can also be present in the compositions.

Formulations of the compositions of the invention include those suitable for intradermal, inhalation, oral/nasal, topical, ophthalmic, and/or parenteral administration. The formulations may conveniently be presented in unit dosage form and may be prepared by any methods well known in the art of pharmacy. The amount of active ingredient (e.g., VIP or a nucleic acid encoding VIP and the one or more one or more agents useful for treating Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection) which can be combined with a carrier material to produce a single dosage form will vary depending upon the host being treated, the particular mode of administration, e.g., ophthalmic, topical, intraperitoneal, nasal, oral, subcutaneous, intravenous, or intaarterial administration. The amount of active ingredient which can be combined with a carrier material to produce a single dosage form will generally be that amount of the active ingredients which produces a therapeutic effect (e.g., one or more of any of the therapeutic effects described herein).

Pharmaceutical formulations of this invention can be prepared according to any method known to the art for the manufacture of pharmaceuticals. Such drugs can contain sweetening agents, flavoring agents, coloring agents, and preserving agents. A formulation can be admixed with nontoxic pharmaceutically acceptable excipients which are suitable for manufacture. Formulations may comprise one or more diluents, emulsifiers, preservatives, buffers, excipients, etc., and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders, such as lactose or starches, lubricants, such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., VIP or a nucleic acid encoding a VIP and one or more agents useful for treating Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection, in any combination) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth, and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long-chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents, and one or more sweetening agents, such as sucrose, aspartame, or saccharin. Formulations can be adjusted for osmolarity.

In some embodiments, oil-based pharmaceuticals are used for administration. Oil-based suspensions can be formulated by suspending active agents in a vegetable oil, such as arachis oil, olive oil, sesame oil, or coconut oil, or in a mineral oil, such as liquid paraffin; or a mixture of these. See, e.g., U.S. Pat. No. 5,716,928, describing using essential oils or essential oil components for increasing bioavailability and reducing inter- and intra-individual variability of orally administered hydrophobic pharmaceutical compounds (see also, U.S. Pat. No. 5,858,401). The oil suspensions can contain a thickening agent, such as beeswax, hard paraffin, or cetyl alcohol. Sweetening agents can be added to provide a palatable oral preparation, such as glycerol, sorbitol, or sucrose. These formulations can be preserved by the addition of an antioxidant such as ascorbic acid. As an example of an injectable oil vehicle, see Minto, J. Pharmacol. Exp. Ther. 281:93-102, 1997.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally-occurring phosphatides, such as soybean lecithin, esters, or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate, and/or an ethoxylated sorbitan trioleate.

The pharmaceutical compounds can also be administered by in intranasal or intraocular routes including insufflation, powders, and aerosol formulations (for examples of steroid inhalants, see e.g., Rohatagi, J. Clin. Pharmacol. 35:1187-1193, 1995; Tjwa, Ann. Allergy Asthma Immunol. 75:107-111, 1995). For example, the pharmaceutical compounds can be delivered as nanoparticles or can be administered by scleral diffusion or subconjunctival injection. The pharmaceutical compounds can be administered using any of the exemplary routes of administration described herein.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres (e.g., nanoparticles) for slow release in the body. For example, microspheres can be administered via intradermal injection of drug which slowly release subcutaneously; see Rao, J. Biomater Sci. Polym. Ed. 7:623-645, 1995; as biodegradable and injectable gel formulations, see, e.g., Gao, Pharm. Res. 12:857-863, 1995; or, as microspheres for oral administration, see, e.g., Eyles, J. Pharm. Pharmacol. 49:669-674, 1997.

In some embodiments, a VIP or a nucleic acid encoding a VIP is administered to the patient as a composition that includes a non-natural molecule or compound (e.g., a polymer). In some embodiments, a VIP or a nucleic acid encoding a VIP is administered to the patient as a composition that is formulated using any of the carriers, excipients, or forms described herein. F For example, a VIP or a nucleic acid encoding a VIP can be administered in a composition that is formulated using a colloidal carrier and/or formulated as a composition containing liposomes, niosomes, nanoparticles (e.g., synthetic organic nanoparticles), and microemulsions. In some embodiments, a subject is administered a composition that includes a VIP or a nucleic acid that encodes a VIP, and a polymer (e.g., a polyvinyl alcohol or a polyethylene glycol).

In some embodiments, the pharmaceutical compound includes a non-natural molecule or compound (e.g., a polymer). In some embodiments, the pharmaceutical composition is formulated using a colloidal carrier and/or formulated as a composition containing liposomes, niosomes, nanoparticles (e.g., synthetic organic nanoparticles), and microemulsions. In some embodiments, a subject is administered a pharmaceutical composition that includes a VIP or a nucleic acid that encodes a VIP, and a polymer (e.g., a polyvinyl alcohol or a polyethylene glycol). Any of the pharmaceutical compositions described herein can include a polymer (e.g., a polyvinyl alcohol or a polyethylene glycol).

Any of the compositions containing a VIP or a nucleic acid encoding a VIP can, e.g., contain one or more of: hydroxypropyl methylcellulose, carboxy methylcellulose, polyvinyl alcohol, carbopol, polyvinyl pyrrolidone, polyethylene glycol, dextran, hyaluronic acid, carbomer 940, HP-Guar, and benzalkonium chloride. Any of the pharmaceutical compositions described herein can, e.g., contain one or more of: hydroxypropyl methylcellulose, carboxy methylcellulose, polyvinyl alcohol, carbopol, polyvinyl pyrrolidone, polyethylene glycol, dextran, hyaluronic acid, carbomer 940, HP-Guar, and benzalkonium chloride.

In some embodiments, the pharmaceutical compounds can be parenterally administered, such as by intravenous (IV), intramuscular, intraperitoneal, or subcutaneous administration, or administration into a body cavity, a lumen of an organ, or into the cerebrospinal fluid of a subject. These formulations can comprise a solution of active agent dissolved in a pharmaceutically acceptable carrier. Acceptable vehicles and solvents that can be employed are water and Ringer's solution, or an isotonic sodium chloride. In addition, sterile fixed oils can be employed as a solvent or suspending medium. For this purpose any bland fixed oil can be employed including synthetic mono- or diglycerides. In addition, fatty acids, such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate, and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous (e.g., substantially uninterrupted introduction into a blood vessel for a specified period of time).

In some embodiments, the pharmaceutical compounds and formulations can be lyophilized. Stable lyophilized formulations comprising VIP and one or more agents useful for treating Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection can be made by lyophilizing a solution comprising VIP or a nucleic acid encoding VIP, one or more agents useful for treating Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection and a bulking agent, e.g., mannitol, trehalose, raffinose, and sucrose, or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5, but less than 6.5. See, e.g., US2004/0028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400 and 6,007,839; Al-Muhammed, J. Microencapsul. 13:293-306, 1996; Chonn, Curr. Opin. Biotechnol. 6:698-708, 1995; and Ostro, Am. J. Hosp. Pharm. 46:1576-1587, 1989.

The formulations of the invention can be administered for prophylactic and/or therapeutic treatments. In some embodiments, for therapeutic applications, compositions are administered to a subject who is at risk of or has a disorder described herein, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to reduce the number of symptoms or reduce the severity, duration, and/or frequency of one or more symptoms of Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection in a subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age, and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones, J. Steroid Biochem. Mol. Biol. 58:611-617, 1996; Groning, Pharmazie 51:337-341, 1996; Fotherby, Contraception 54:59-69, 1996; Johnson, J. Pharm. Sci. 84:1144-1146, 1995; Rohatagi, Pharmazie 50:610-613, 1995; Brophy, Eur. J. Clin. Pharmacol. 24:103-108, 1983; Remington: The Science and Practice of Pharmacy, 21st ed., 2005). The state of the art allows the clinician to determine the dosage regimen for each individual patient, the active agents, and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, and the like. The formulations should provide a sufficient quantity of the active agents to effectively treat, prevent or ameliorate conditions, diseases, or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray, or inhalation. Actual methods for preparing parenterally or non-parenterally administrable formulations will be known or apparent to those skilled in the art and are described in more detail in such publications as Remington: The Science and Practice of Pharmacy, 21st ed., 2005.

The invention is further illustrated by the following examples. The examples are provided for illustrative purposes only. They are not to be construed as limiting the scope or content of the invention in any way.

### EXAMPLE

### Example 1

### Nerve Loss-Related Corneal Endothelial Cell Loss and Ability of VIP to Reduce Nerve Loss-Related Corneal Endothelial Cell Loss

A set of experiments was performed to evaluate corneal endothelial cell alterations after trigeminal axotomy and the effect of VIP on corneal endothelial cells after trigeminal axotomy.

### Materials and Methods

### Animals and Surgical Procedure

Six- to eight-week old male BALB/c mice (Charles River, Wilmington, Mass.) were used in these experiments. Trigeminal axotomy was performed by first anesthetizing the animals with a ketamine (100 mg/mL)/xylazine (20 mg/mL)/acepromazine (15 mg/mL) mixture. After anesthetization, small incision lateral canthotomy was performed, two tractional sutures were placed on the lid skin, and the conjunctival fornix were incised circumferentially around 90 degrees. The eye globe was rotated nasally by gently pushing the nasal fornix with blunt forceps, exposing the trigeminal nerve and minimizing intraoperative bleeding through mild elevation of the intraorbital pressure. The ophthalmic branches of the trigeminal nerve were cut at the posterior sclera close to the optic nerve with sharp forceps. After cutting the branches of the trigeminal nerve, the skin was sutured using 8-0 nylon. Tarsorrhaphy was performed to reduce the risk of infection and exposure keratitis in the mice. Finally, an antibiotic ointment (bacitracin-neomycin-polymyxin mixture) was applied to the sutured area and the treated eye in the mice. Fluxinin (0.25 mg/kg body weight) was administered every 12 hours for 24 hours by subcutaneous injection. The sham surgery was conducted by repeating the steps of above procedures, except for the nerve-cutting step.

### Immunoprecipitation

After fixation by acetone, the mouse corneas were permeabilized and blocked in 2% bovine serum albumin (BSA). The corneas were stained with primary antibody (rabbit anti-zonula occludens-1 (ZO-1) protein, 1:200, Invitrogen, Grand Island, N.Y.) overnight in 4° C. and incubated with secondary antibody (fluorescein isothiocyanate (FITC) anti-rabbit, 1:200, Santa Cruz Biotechnology, Santa Cruz, Calif.) for one hour at room temperature. The corneas were then stained with monoclonal anti-β-tubulin antibody (anti-neuron-specific beta-3 tubulin-NL637, 1:100, R & D Systems Inc., Minneapolis, Minn.) overnight. TO-PRO-3 iodide (Molecular Probes, Eugene, Oreg.) was used to stain the nuclei. Anti-VIP rabbit antibody (ab78536, 1:100 in PBS, Abcam Inc., Cambridge, Mass.) and a secondary antibody FITC-conjugated donkey anti-rabbit IgG (711-095-152, Jackson ImmunoResearch Laboratories Inc., West Grove, Pa.) were used for VIP immunohistochemistry. Digital images were obtained from central and peripheral endothelial cells using a spectral photometric confocal microscope (Leica DM6000S with LCS 1.3.1 software, Solms, Germany). The whole thickness of the corneas were imaged to evaluate the central and peripheral nerves and VIP distribution in the cornea at the z-axis steps of 2 μm using confocal microscope (FV 10-ASW, Olympus, Tokyo, Japan).

### Anterior Segment Optical Coherence Tomography

Normal and post-operative cornea were examined using anterior segment optical coherence tomography (AS-OCT, RTVue, Optoview, Inc., Fremont, Calif.) at 7, 14 and 21 days after the surgical procedure. AS-OCT is fast imaging system with high tissue resolution of 5 μm. The mice were placed in front of the AS-OCT machine and gently held by hand, taking care not to elevate the intraocular pressure in the mice. All images were taken at least twice in all mice until the clear images could be obtained, to confirm the reproducibility of the data. Images were taken in raster scan mode, with 0.2-mm steps within the diameter of 4 mm. The corneal thickness was measured using built-in software.

### VIP Measurement

The levels of VIP mRNA in the treated corneas of the mice was determined by quantitative reverse-transcriptase PCR using standard methods known in the art. In these experiments, total RNA was isolated from the individual corneas of normal mice and mice after trigeminal axotomy.

The levels of VIP were determined by using a competitive enzyme immunoassay (EIA). In these experiments, whole corneas were first excised in normal mice and treated mice at 7 and 14 days after trigeminal axotomy (n=5, per group per time point, two experiments=30 mice). The VIP levels in the cornea were determined using an EIA kit (Penninsula Laboratories). The individual corneas were collected and homogenized mechanically in 250 μL of normal saline. The samples were centrifuged at 5000×g for 10 minutes and the aliquot of each supernatant was assayed in triplicate for VIP according to the manufacturer's instruction. The assay sensitivity was 2-3 pg/mL. The results are expressed as average pg of VIP per mL.

### VIP Treatment

BALBc mice after trigeminal axotomy received daily intraperitoneal (IP) injections of VIP, 5 nM in 10 μL (VIP; Bachem Americas, Inc., Torrance, Calif.) beginning from the day of surgery until in vivo AS-OCT images were taken under anesthesia and the corneas were harvested on day 14. The control mice were similarly injected with sterile saline.

### Data Analysis and Statistics

Corneal endothelial cells were counted using cell counter plug-ins with ImageJ software (NIH, Bethesda, Md.). The statistical analyses were performed by SPSS 16.0. Independent Student t-tests were used to compare the normal and axotomized corneas. The results are expressed as mean±SEM, and considered significant if p<0.05.

Image J 1.45 and Neuron J were used to create stacked images and to calculate nerve density. Neuron J is an Image J plugin software to facilitate the tracing and quantification of elongated image structure (see, the website at imagescience.org/meijering/software/neuronj/). All of the nerve branches of stacked images of immunofluorescent histology were traced using Neuron J software. Neuron J measured the total length of the traced nerve, then the total nerve lengths were converted to nerve density (by dividing the total nerve length by its area). The success rate of treatment was determined up to day 14 postoperatively, based on the images of immunofluorescent staining. If the normal nerve was observed even partially, it was regarded as failure of the surgery and excluded from the analysis of nerve density. The data were analyzed using statistical analysis software (SSRI Co. Ltd., Tokyo, Japan). The unpaired Student's t test was used to compare the nerve density between normal and postoperative corneas. For each test, differences were considered significant at P value of less than 0.05 and represented as mean +/−standard deviation.

### Results

The survival rate of the treatment was 100% without any systemic complications, including neurologic complications like paralysis. The blink reflex diminished or reduced from day 1. No postoperative ocular complications, such as cataract, infectious keratitis, and phthisis were observed.

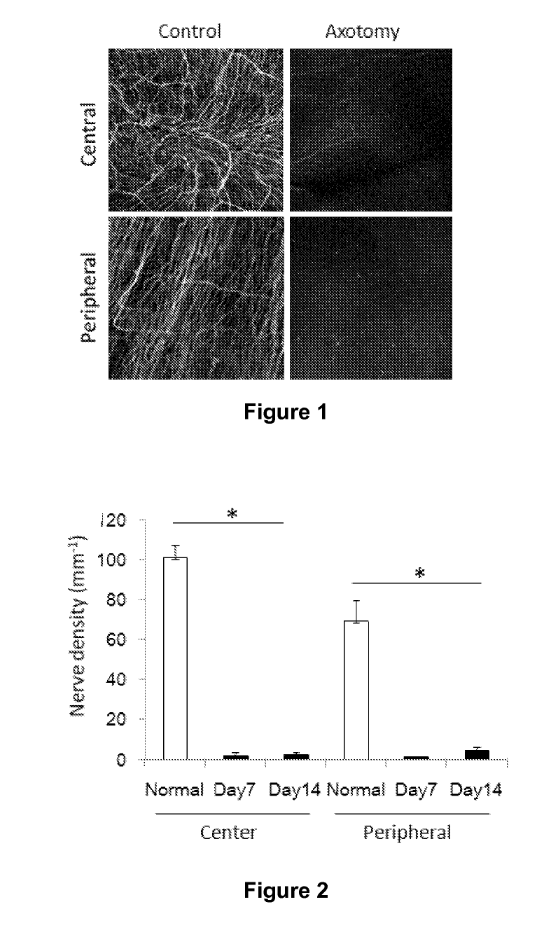

The corneal nerve decreased after the surgical procedure. In the axotomized eyes, subbasal nerve plexus completely diminished from day 1, both in the center and peripheral cornea, even in mice without epithelial defects (FIG. 1). The stromal nerve apparently decreased and the residual nerve branches became dotty, fragmented, narrowed or swollen, and straight from (FIG. 1). FIG. 2 shows the alteration in corneal nerve density in the axotomized eye in the center and peripheral cornea. The nerve densities decreased from 115.9±12.6 (central) and 106.2±8 0 mm/mm² (peripheral) in normal cornea, to 10.9±7.7 (central) and 13.3±0 2 mm/mm² (peripheral) in the axotomized cornea at day 1 (p<0.001).

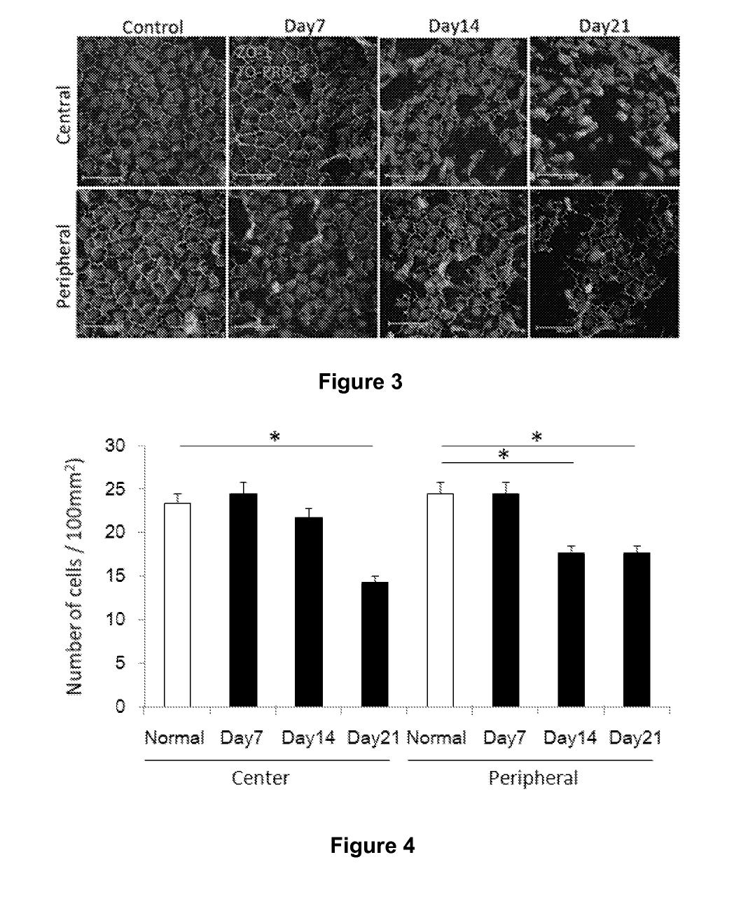

To investigate the endothelial cells response to trigeminal nerve axotomy, immunofluorescence was performed to determine and the densitometry of the corneal endothelial cells was compared on days 7, 14 and 21 after axotomy. FIG. 3 shows confocal images of central (top row) and peripheral (bottom row) corneal endothelial cells. Of interest, trigeminal nerve axotomized corneas demonstrate an obvious decrease in the number of both central and peripheral corneal endothelial cells. The corneal endothelial cells start to lose hexagonal shape and become somehow larger at day 7. Corneal endothelial cell density (ECD) significantly decreased on days 14 and 21 in the peripheral cornea and on day 21 in the center cornea (FIG. 4; p<0.05).

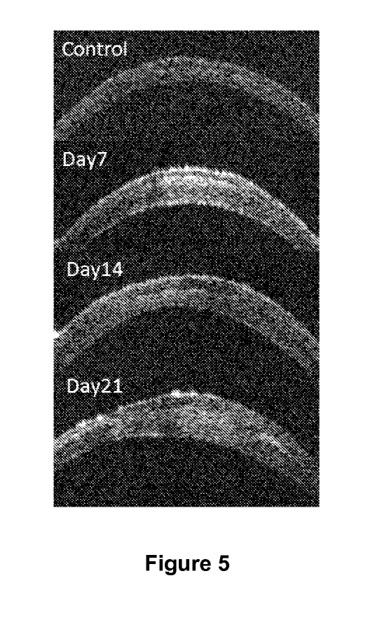

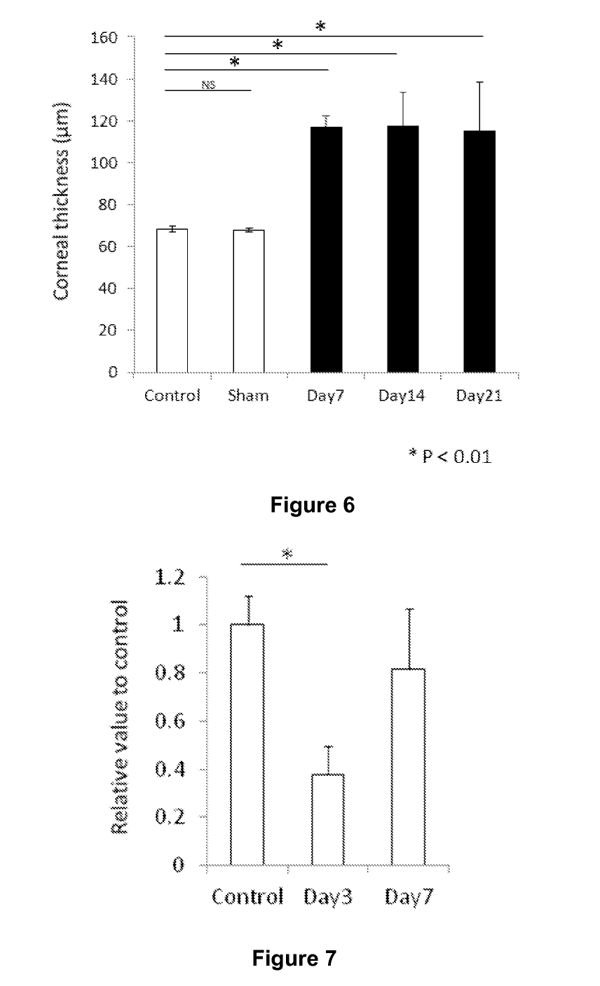

AS-OCT revealed an increase in corneal thickness after trigeminal axotomy (FIGS. 5 and 6). There were statistical significant differences in corneal thickness between normal and post-trigeminal axotomy mice from day 7 (P<0.01).

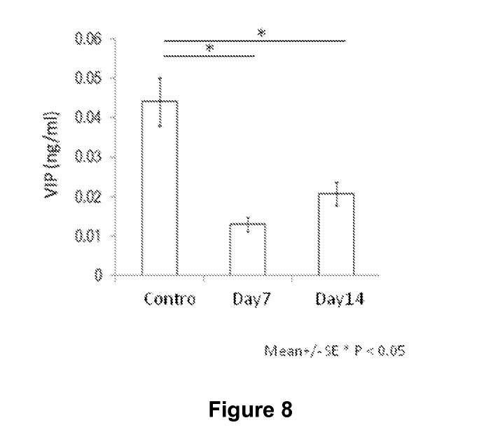

The mRNA level of VIP was significantly lower at day 3 post-trigeminal mice than the normal control mice (FIG. 7; p<0.05). The protein expression pattern for VIP in the cornea in mice after trigeminal axotomy was assessed and compared to the corneas of control mice. VIP levels significantly decreased from day 7 in mice after trigeminal axotomy and remained at a low level (FIG. 8; p<0.05).

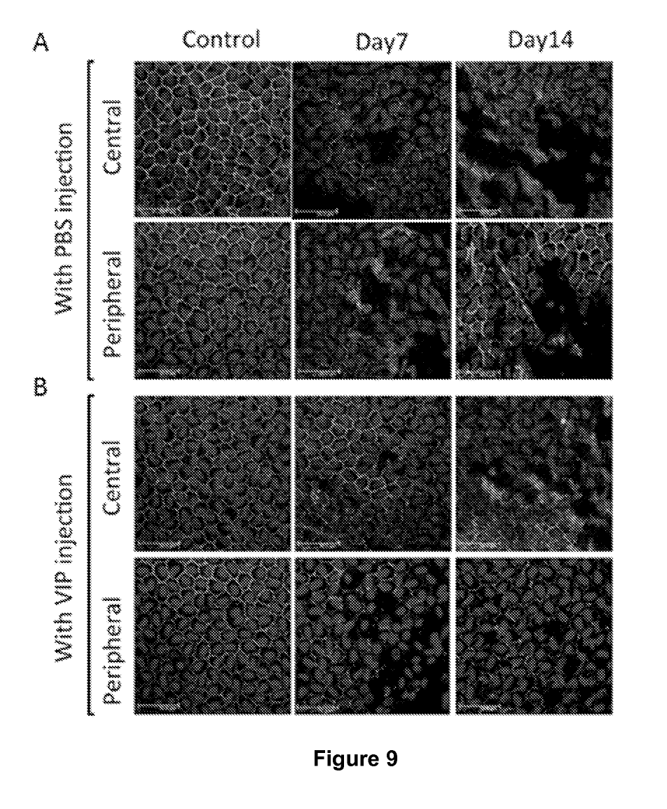

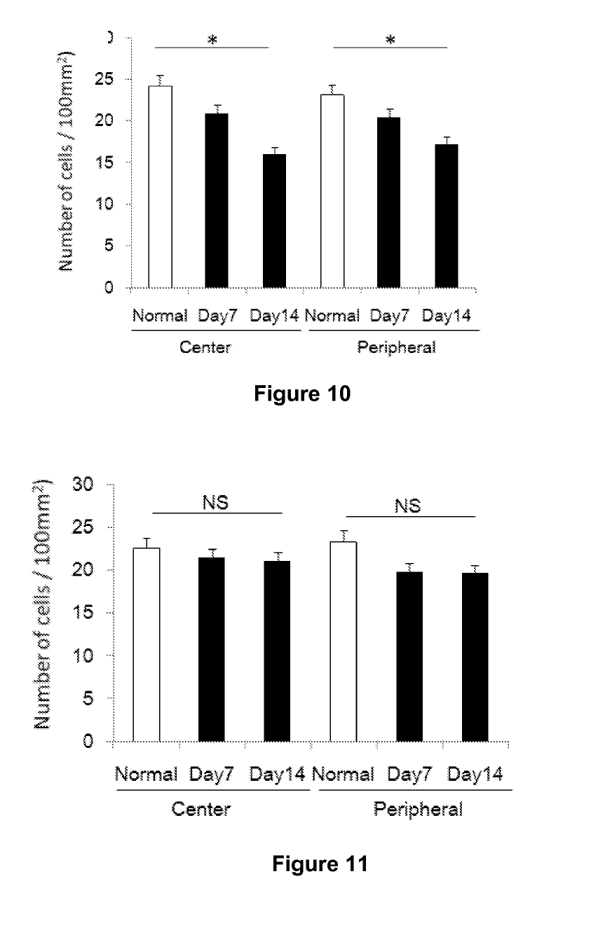

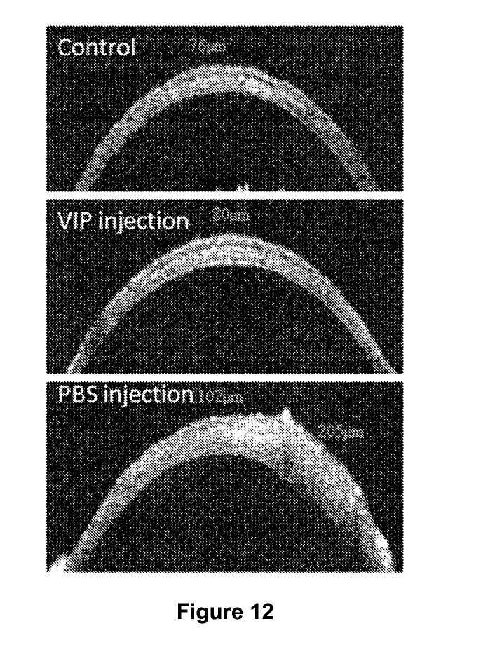

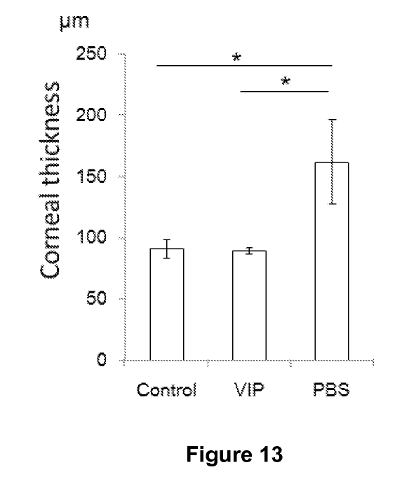

In the VIP-treated group, the corneal endothelial cell density didn't decrease in the mice after trigeminal axotomy, while the corneal endothelial cell density significantly decreased in mice after trigeminal axotomy that received only saline at day 14 (FIGS. 9A, 9B, 10, and 11). The corneal thickness in the saline-treated group significantly increased after trigeminal axotomy, whereas the corneal thickness remained within the normal ranged in the VIP-treated group (FIGS. 12 and 13).

In sum, these data show that there are significantly diminished numbers of corneal endothelial cells after trigeminal nerve axotomy, which appears soon after the nerve plexus diminishes. The data indicate the protective role of the trigeminal nerve in maintaining endothelial cells, and that VIP can protect against corneal endothelial cell loss (e.g., nerve loss-related corneal endothelial cell loss) in mammals.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of reducing nerve loss-related corneal endothelial cell loss in a subject, the method comprising:
selecting a subject identified as having an eye with reduced numbers of corneal nerves as compared to a reference eye; and
administering vasoactive intestinal peptide (VIP) to the selected subject.

2. The method of claim 1, wherein the VIP is topically administered to the eye of the subject.

3. The method of claim 1, wherein the VIP is administered to the eye of the subject by systemic administration, subconjunctival injection, or intraperitoneal injection.

4. The method of claim 1, wherein the subject has Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection.

5. The method of claim 1, wherein the subject has Fuchs' endothelial corneal dystrophy.

6. The method of claim 1, wherein the subject has been diagnosed as having Fuchs' endothelial corneal dystrophy, pseudophakic bullous keratopathy, keratoconus, pseudoexfoliation syndrome, atopic keratoconjunctivitis, herpetic stromal keratitis, endothelial cell loss after full-thickness or partial-thickness corneal transplantation, herpes zoster ophthalmicus, uveitis, or graft rejection.

7. The method of claim 6, wherein the subject has been diagnosed as having Fuchs' endothelial corneal dystrophy.

8. The method of claim 1, further comprising identifying a subject as having an eye with reduced numbers of corneal nerves as compared to a reference eye.

9. The method of claim 8, wherein the identifying is performed using in vivo confocal microscopy.

10. The method of claim 4, wherein said administering results in treatment of Fuchs' endothelial corneal dystrophy, pseudophakic bullous keratopathy, pseudoexfoliation syndrome, herpetic stromal keratitis, endothelial cell loss after full-thickness or partial-thickness corneal transplantation, herpes zoster ophthalmicus, uveitis, or graft rejection in the subject.

11. The method of claim 1, wherein the subject is administered two or more doses of VIP.

12. The method of claim 11, wherein the two or more doses are administered to the subject at a frequency of at least once a month.

13. The method of claim 12, wherein the two or more doses are administered to the subject at a frequency of at least once every two weeks.

14. The method of claim 13, wherein the two or more doses are administered to the subject at a frequency of at least once every week.

15. The method of claim 1, wherein the reference eye is an eye of a healthy control.

16. The method of claim 8, wherein the reference eye is an eye of a healthy control.
